(19) United States
(12) Patent Application Publication
(10) Pub. No.: US 2019/0037812 A1
(43) Pub. Date: Feb. 7, 2019

(54) DEVICE AND METHOD FOR MONITORING USING DRONE

(71) Applicant: Samsung Electronics Co., Ltd., Suwon-si (KR)

(72) Inventors: Joonseong KANG, Suwon-si (KR); Sang Joon KIM, Hwaseong-si (KR); Young Jun HONG, Seoul (KR)

(73) Assignee: Samsung Electronics Co., Ltd., Suwon-si (KR)

(21) Appl. No.: 15/992,290

(22) Filed: May 30, 2018

(30) Foreign Application Priority Data
Aug. 7, 2017 (KR) 10-2017-0099494

(51) Int. Cl. A01K 29/00; G05D 1/00; B64C 39/02; A61B 5/07; A61B 5/00; A61B 5/11

(52) U.S. Cl. CPC A01K 29/005; A61B 2503/40; G05D 1/0088; B64C 39/024; A61B 5/076; A61B 5/6869; A61B 5/6871; A61B 5/6873; A61B 5/1116; A61B 5/7221; A61B 5/7282; B64C 2201/141; B64C 2201/146; B64C 2201/12; G05D 1/0022

(57) ABSTRACT

Disclosed is a monitoring device including a first measurement device disposed inside an organ of a target, the first measurement device being configured to measure a first biosignal of the target and to transmit the first biosignal, a second measurement device disposed inside a skin tissue of the target, and the second measurement device being configured to measure a second biosignal of the target, to receive the first biosignal, and to transmit bioinformation comprising the first biosignal and the second biosignal, a drone configured to move to an area in which the target is positioned, to receive the bioinformation from the second measurement device, and to transmit the bioinformation, and an analyzer configured to monitor and analyze a condition of the target based on the bioinformation received from the drone.

### CROSS-REFERENCE TO RELATED APPLICATIONS

This application claims the benefit under 35 USC § 119(a) of Korean Patent Application No. 10-2017-0099494 filed on Aug. 7, 2017 in the Korean Intellectual Property Office, the entire disclosure of which is incorporated herein by reference for all purposes.

### BACKGROUND

### 1. Field

The following description relates to a device and method for monitoring livestock using a drone.

### 2. Description of Related Art

As the livestock industry has developed, it has become difficult to thoroughly check health conditions or other conditions of a large number of livestock, such as, for example, cows, pigs, and sheep. The delay in checking the condition of the livestock results in a delay in treatment for abnormal conditions. Even when treatment for infectious diseases is urgently needed, action may be delayed due to delay in recognizing the condition.

For example, body temperatures of livestock such as cows may vary in response to abnormal health conditions. In an example, a cow suffering from indigestion or enteritis has a temperature that is 0.5 to 1 degree lower than a normal temperature of 39 degrees. In another example, a cow suffering from pneumonia or noxious heat has a temperature that is 0.5 to 1 degree higher than a normal temperature of 39 degrees. Thus, monitoring a temperature of a cow indicates a health of the cow. However, it is difficult to detect a change in a condition of a cow in case where a large number of livestock are being raised or pastured, and it is not easy to collect or gather monitoring information on a large number of livestock.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, there is provided a monitoring device, including a first measurement device disposed inside an organ of a target, the first measurement device being configured to measure a first biosignal of the target and to transmit the first biosignal, a second measurement device disposed inside a skin tissue of the target, and the second measurement device being configured to measure a second biosignal of the target, to receive the first biosignal, and to transmit bioinformation comprising the first biosignal and the second biosignal, a drone configured to move to an area in which the target is positioned, to receive the bioinformation from the second measurement device, and to transmit the bioinformation, and an analyzer configured to monitor and analyze a condition of the target based on the bioinformation received from the drone.

The target may include at least one of a livestock comprising a cow, a pig, a sheep, a goat, or a horse, or a pet comprising a bird, a dog or a cat.

The organ of the target may include at least one of a stomach, a heart, a kidney, an udder, a small intestine, a large intestine, or a rectum.

The second measurement device may be configured to transmit the bioinformation based on an upward directivity of the target.

The second measurement device may include a second sensor configured to measure the second biosignal, and a second communication interface configured to receive at least one of the first biosignal or a detection signal transmitted by the drone, and to transmit the bioinformation.

The second measurement device may be configured to transmit the bioinformation to the drone, in response to receiving a detection signal transmitted by the drone.

The first measurement device and the second measurement device may be configured to communicate with each other based on any one or any combination of a human body communication method using a tissue of the target as a medium, a communication method using a 433 megahertz (MHz)-medical implant communication service (MICS) frequency band, or a near field communication (NFC) method.

The second measurement device and the drone may be configured to communicate with each other based on any one or any combination of a communication method using a 2.4 gigahertz (GHz)-industrial, scientific, and medical (ISM) frequency band including a Bluetooth and a Zigbee, a communication method using a 433 megahertz (MHz)-medical implant communication service (MICS) frequency band, or a near field communication (NFC) method.

The drone may be configured to recognize identification information and a position of the target based on whether the bioinformation is received in the area in which the target is positioned.

The drone may be configured to match the identification information and the position of the target to the bioinformation and to transmit the matched bioinformation to the analyzer.

The drone may be configured to transmit a detection signal to verify a presence of the second measurement device.

The drone may be configured to iteratively transmit the detection signal to verify a presence of the second measurement device in the area in which the target is positioned, in response to not receiving the bioinformation.

A movement of the drone may be controlled by a control signal from an outside of the monitoring device.

The drone may move in an area based on any one or any combination of a regular pattern or a random pattern.

The analyzer may be configured to estimate a representative biosignal of the target based on the bioinformation and to monitor a health condition of the target based on the estimated representative biosignal.

The analyzer may be configured to determine the first biosignal as the representative bioinformation of the target, in response to the first biosignal being within an error range associated with the first biosignal, and determine the second biosignal as the representative bioinformation of the target, in response to the first biosignal being outside the error range.

The analyzer may be provided integrally with the drone.

The analyzer may be provided separate from the drone.

The first measurement device may include a first sensor configured to measure the first biosignal, and a first communication interface configured to transmit the first biosignal to the second measurement device.

The monitoring device may include a drone controller configured to control an operation including a movement of the drone.

In another general aspect, there is provided an method of monitoring device a target, the method including moving a drone to an area in which the target is positioned, transmitting a detection signal, by the drone, to verify a presence of a second measurement device in the area, receiving bioinformation comprising a first biosignal and a second biosignal from the second measurement device, in response to the detection signal being received by the second measurement device, the first biosignal being measured by a first sensor disposed inside an organ of the target and the second biosignal being measured by a second sensor disposed inside a skin tissue of the target, and monitoring a condition of the target based on the bioinformation.

Other features and aspects will be apparent from the following detailed description, the drawings, and the claims.

### BRIEF DESCRIPTION OF THE DRAWINGS

FIG. 1 illustrates an example of a monitoring device.

FIG. 2 illustrates an example of an environment in which a monitoring device is used.

FIG. 3 is a diagram illustrating an example of each constituent element of a monitoring device.

FIG. 4 is a diagram illustrating an example of a monitoring device.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. The sequences of operations described herein are merely examples, and are not limited to those set forth herein, but may be changed as will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of functions and constructions that are known in the art may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

The following specific structural or functional descriptions are exemplary to merely describe the examples, and the scope of the examples is not limited to the descriptions provided in the present specification. Various changes and modifications can be made after an understanding of the disclosure of this application.

Although terms of “first” or “second” are used to explain various components, the components are not limited to the terms. These terms should be used only to distinguish one component from another component. For example, a “first” component may be referred to as a “second” component, or similarly, and the “second” component may be referred to as the “first” component within the scope of the right according to the concept of the present disclosure. As used herein, the singular forms are intended to include the plural forms as well, unless the context clearly indicates otherwise.

A function or an operation illustrated in a block may be performed not in a sequential order according to examples. For example, functions or operations illustrated in successive blocks may be actually performed concurrently, or an order of the blocks may be changed based on related functions or operations.

Hereinafter, examples will be described in detail below with reference to the accompanying drawings, and like reference numerals refer to the like elements throughout.

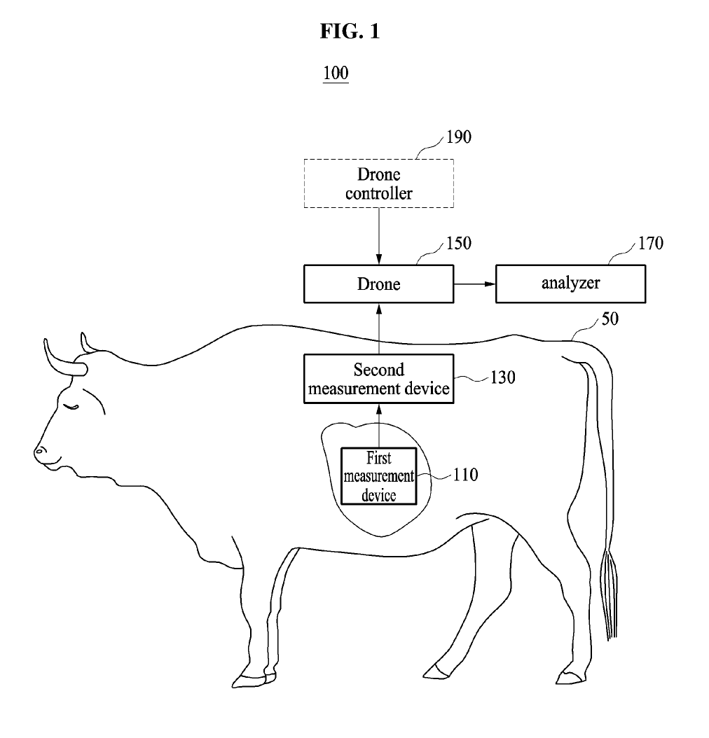

FIG. 1 illustrates an example of a monitoring device. In an example, a monitoring device 100 includes a first measurement device 110, a second measurement device 130, a drone 150, and an analyzer 170. The monitoring device 100 further includes a drone controller 190 configured to control a movement of the drone 150.

In an example, the first measurement device 110 is disposed inside an organ of a target living body 50, and measures a first biosignal of the target living body 50 and wirelessly transmits the first biosignal to the second measurement device 130. In an example, the target living body 50 includes a livestock such as, for example, cow, cattle, buffalo, pig, sheep, goat, chicken, donkey, llama, or horse. In another example, the target living body 50 is a pet such as, a dog, a bird, or a cat. The organ of the target living body 50 includes organs such as, for example, stomach, small intestine, large intestine, heart, kidney, udder, or rectum of the target living body 50. For example, when the target living body 50 is a ruminant who chews cud, such as, for example, a cow and a sheep, the stomach of the target living body 50 may be divided into four or five compartments. Thus, it may be assumed that the organ of the target living body 50 may include four or five stomachs.

In an example, the first measurement device 110 includes a first sensor (not shown) configured to measure the first biosignal and a first communication interface (not shown) configured to wirelessly transmit the first biosignal to the second measurement device 130.

In an example, the first biosignal includes biosignals, such as, for example, a body temperature, an acidity, an oxygen saturation, an electrocardiogram (ECG), a pulse, or a blood flow. The first sensor includes various biosignal detecting sensors, such as, for example, a heat detecting sensor, an acidity detecting sensor, an oxygen saturation measuring sensor, an ECG measuring sensor, a biosensor, and a nucleic acid sensor.

In an example, the second measurement device 130 is disposed inside a skin tissue of the target living body 50, measures a second biosignal of the target living body 50, receives the first biosignal, and transmits bioinformation including the first biosignal and the second biosignal.

The second measurement device 130 includes, for example, a second sensor (not shown) and a second communication interface (not shown). The second sensor may measure the second biosignal. The second communication interface may receive at least one of the first biosignal or a detection signal of the drone 150, and transmit the bioinformation.

In an example, the second biosignal includes biosignals such as, for example, a body temperature, an acidity, an oxygen saturation, an ECG, a pulse, or a blood flow. Similar to the first sensor, the second sensor includes various biosignal detecting sensors, such as, for example, a heat detecting sensor, an acidity detecting sensor, an oxygen saturation measuring sensor, an ECG measuring sensor, a biosensor, and a nucleic acid sensor. The second biosignal may be identical to or differ from the first biosignal.

The second measurement device 130 transmits the bioinformation based on an upward directivity of the target living body 50.

The first measurement device 110 and the second measurement device 130 communicate with each other based on various methods such as, for example, a human body communication method using a tissue of the target living body 50 as a medium, a communication method using a 433 megahertz (MHz)-medical implant communication service (MICS) frequency band, or a near field communication (NFC) method. The human body communication method may transmit data using an electric field generated around a tissue when a voltage is applied to the tissue through a human or an animal, instead of a cable.

The second measurement device 130 transmits the bioinformation to the drone 150 when it receives a detection signal transmitted by the drone 150.

The second measurement device 130 and the drone 150 communicate with each other based on a communication method such as, for example, a 2.4 gigahertz (GHz) industrial, scientific, and medical (ISM) frequency band including a Bluetooth and a Zigbee, a communication method using a 433 megahertz MICS frequency band, or an NFC method.

In an example, the drone 150 moves to an area in which the target living body 50 is positioned, receives the bioinformation from the second measurement device 130, and transmits the bioinformation to the analyzer 170.

In an example, the drone 150 recognizes identification information and a position of the target living body 50 based on whether the bioinformation is received in the area in which the target living body 50 is positioned. In an example, the identification information of the target living body 50 is received from the second measurement device 130 and a tag. In an example, the tag is attached on an ear of a cow or a sheep, or is disposed in a separate area of the target living body 50. The position of the target living body 50 may be acquired based on coordinates of a current position of the drone 150 receiving the bioinformation from the target living body 50. In an example, the coordinates of the current position of the drone 150 is acquired through a global positioning system (GPS) sensor included in the drone 150. The drone 150 may match the identification information and the position of the target living body 50 to the bioinformation, and transmit the matched bioinformation to the analyzer 170.

In an example, the drone 150 may transmit the detection signal to verify whether the second measurement device 130 is present. The drone 150 may iteratively transmit the detection signal for verifying the second measurement device 130 is present in the area in which the target living body 50 is positioned. The drone 150 may iteratively transmit the detection signal when the bioinformation is not receiving during a period of time.

The drone 150 may freely move or the movement of the drone 150 may be controlled by a control signal from an outside of the monitoring device 100. The control signal from the outside of the monitoring device 100 may be received from, for example, the drone controller 190.

The drone 150 may move in a area based on a regular pattern or a random pattern. The drone 150 may move in the area, for example, an inside of a farm and a pen, at a time interval or at random times.

In an example, the analyzer 170 analyzes and monitors a condition of the target living body 50 based on the bioinformation received from the drone 150. In an example, the analyzer 170 estimates representative bioinformation of the target living body 50 based on the bioinformation, and monitors a health condition of the target living body 50 based on the estimated representative bioinformation.

In an example, the analyzer 170 estimates the first biosignal as the representative bioinformation of the target living body 50 when the first biosignal is present within an error range associated with the first biosignal. In an example, the analyzer 170 estimates the second biosignal as the representative bioinformation of the target living body 50 when the first biosignal is out of the error range associated with the first biosignal. The error range may vary depending on a type of each biosignal of the target living body 50. In an example, when a target living body is a cow and a first biosignal is a body temperature, a error range may correspond to a range from −0.5 to +0.5 based on 39 degrees, i.e., a range from 38.5 degrees to 39.5 degrees. In another example, when a target living body is a sheep and a first biosignal is a body temperature, a error range may correspond to a range from −1 to +1 based on 38 degrees to 40 degrees, i.e., a range from 37 degrees to 41 degrees.

For example, when a target living body is a cow, the analyzer 170 estimates a second biosignal, 39.5 degrees, as representative bioinformation of the cow when a first biosignal measured from the cow is 36 degrees and the second biosignal is 39.5 degrees.

In an example, the analyzer 170 is provided integrally with the drone 150, or separate from the drone 150.

The analyzer 170 may include a communication interface (not shown) configured to receive the bioinformation from the drone 150, a memory (not shown) configured to store the bioinformation, and a processor (not shown) configured to analyze and monitor the condition of the target living body 50 based on the bioinformation.

In an example, the drone controller 190 controls an operation including a movement of the drone 150. In an example, the drone controlling apparatus 190 includes a processor configured to generate a control signal for controlling the movement of the drone 150 and a communication interface configured to transmit the control signal. In an example, the drone controller 190 generates the control signal based on a user input. In another example, the drone controller 190 generates the control signal based on a preset program or algorithm.

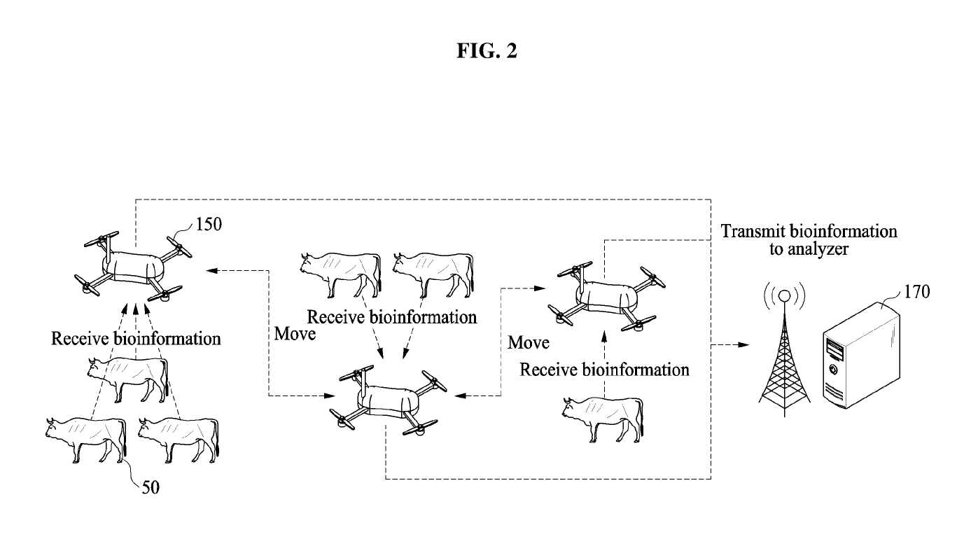

Description of an example of environment in which the monitoring device 100 is used is provided with reference to FIG. 2.

FIG. 2 illustrates that the drone 150 is approaching over the cows 50 in a farm and transmits bioinformation received from the cows 50 to the analyzer 170. Hereinafter, a cow is illustrated as an example of a target living body for ease of description. However, the target living body is not limited to a cow. The target living body includes various livestock and pets.

When the drone 150 moves toward the cows 50 in a farm or on a pen, a second measurement device disposed under a skin tissue of, for example, upper scapular and vertebrae, of each of the cows 50 transmits the bioinformation to the drone 150. A first measurement device including a first sensor may be disposed inside an organ, for example, a second stomach, of each of the cows 50, and measure a first biosignal, for example, a body temperature, an acidity, an oxygen saturation, an electrocardiogram (ECG), a pulse, and a blood flow of a cow. The first measurement device may wirelessly transmit the first biosignal to the second measurement device.

A measurement device, for example, a temperature measurement device, attached outside a body may increase a number of measurement errors of a biosignal because of the affect of an outside environment, for example, a weather and a temperature. In addition, a measurement device, for example, a temperature measurement device, attached outside a body may frequently affect the biosignal measured by a movement of a cow. In particular, a skin temperature of a cow may have a greater variation (standard deviation) than that of a deep body portion temperature of the cow. Because a difference between a temperature of a cow in an abnormal condition and a temperature of the cow in a normal condition is from −1 degree to +1 degree and the difference is relatively small, the measurement device attached outside the body may inaccurately distinguish the difference between the normal condition and the abnormal condition of the cow. In an example, when a measurement device is inserted into a body of the cow, for example, into an organ and a skin tissue of a cow, a measurement error of a biosignal resulting from the outside environment is reduced and the damage to the biosignal is also reduced.

The first measurement device accurately measures a temperature of each cow based on an error ranging from −0.5 degree to +0.5 degree. However, because the first measurement device is included in the body portion of each cow, a signal may be attenuated up to 110 decibel (dB) due to a body of each cow when temperature information is transmitted outside the body. The first measurement device minimizes the signal attenuation resulting from the body of each cow by transmitting the measured temperature information to the second measurement device disposed under the skin tissue of the upper scapular or the vertebrae of each cow. A temperature measured from the inside of stomach using the first measurement device is relatively accurate, but an error in the temperature may increase when a cow eats food or drinks water, in response to the temperatures of the food and the water being greater than or less than the body temperature of the cow, or in response to the food and the water remaining in the stomach for a relatively long period of time.

To reduce such error, in an example, the second measurement device receives the first biosignal measured by the first measurement device within the period of time before feeding each cow.

In an example, the second measurement device is disposed in a space under the skin tissue, for example, upper scapular or vertebrae, of each cow. The upper scapular has a relatively wide concave space such that a device is easily inserted into the upper scapular and an external influence may be relatively small.

The second measurement device may measure the second biosignal, for example, a body temperature, an acidity, an oxygen saturation, an ECG, a pulse, and a blood flow, of each cow from under the skin tissue of, for example, upper scapular or vertebrae, of each cow. The second measurement device may wirelessly transmit, to the drone 150, the bioinformation including the first biosignal measured by the first measurement device and the second biosignal measured by the second measurement device.

For example, the second measurement device measures the body temperature of each cow based on an error ranging from −1 degree to +1 degree. In an example, because the second measurement device is disposed under the skin tissue, the signal may be attenuated due to the skin tissue such that a distance between the second measurement device and an analyzer receiving the bioinformation should be less than or equal to 5 meters.

In general, a distance less than or equal to 5 meters may be an insufficient communication distance in consideration of a size of a farm or a pen. To overcome such insufficient distance, a transmitting device including a relatively large antenna or a large battery may be provided. In another example, to transmit the bioinformation transmitted by the second measurement device to the analyzer, a space of the pen may be remodeled to allow the cows to always pass through a area in the pen and a device for receiving the bioinformation may be disposed in this area to receive bioinformation transmitted from the cows. Thus, a cost of remodeling the pen and installing a passage may increase because the pen needs to be remodeled to allow the cows to pass through a passage.

Because the drone 150 moves freely in an area in the pen, the bioinformation of all cows in the pen may be collected without remodeling the pen. In addition, the drone 150 may collect the bioinformation by approaching each of the cows scattered in a wide area, for example, a farm. Because the drone 150 approaches an object as close as needed regardless of a number of objects in the pen, the second measurement device does not need a relatively large antenna for increasing a transmission efficiency or a large battery for increasing a transmission intensity. The second measurement device may be provided in a relatively small size appropriate for an implant in proportion to a size of each target living body.

A performance of transmitting a signal (bioinformation) from the second measurement device to the drone 150 is maximized by designing a directivity of the signal (bioinformation) such that the signal (bioinformation) is concentrated in a direction from upper scapular or vertebrae of cows toward the sky.

As described above, the drone 150 that collects the bioinformation of each cow in the pen or on the farm may transmit the collected bioinformation to the analyzer 170. The analyzer 170 accurately estimate the bioinformation, for example, a body temperature of each cow, based on the bioinformation collected by the drone 150 and monitors a condition of each cow for 24 hours.

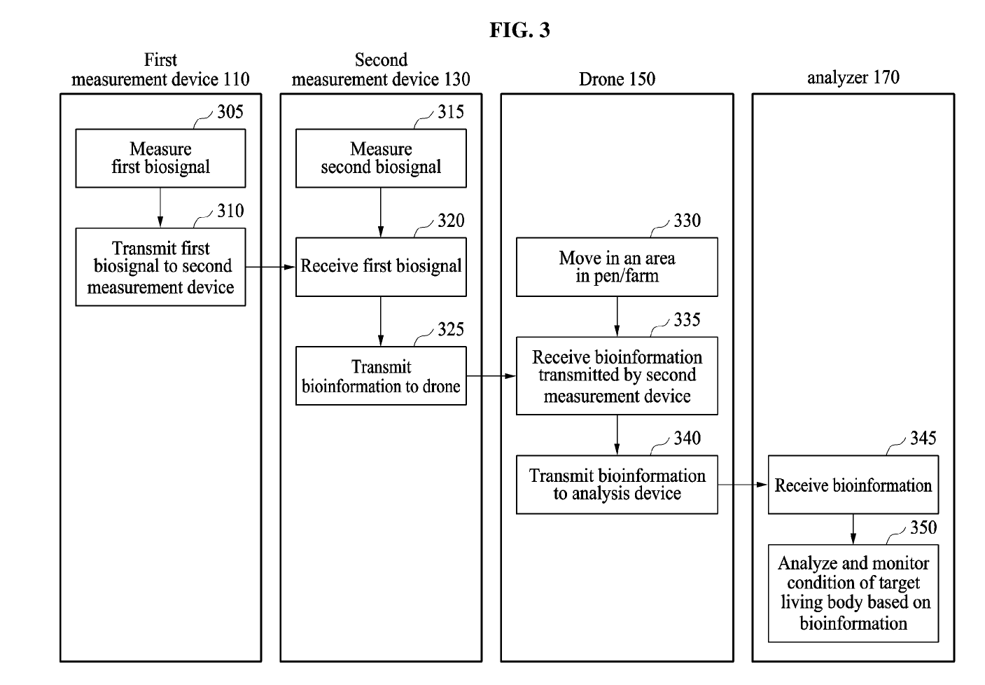

FIG. 3 is a diagram illustrating an example of each constituent element of a monitoring device. The operations in FIG. 3 may be performed in the sequence and manner as shown, although the order of some operations may be changed or some of the operations omitted without departing from the spirit and scope of the illustrative examples described. Many of the operations shown in FIG. 3 may be performed in parallel or concurrently. One or more blocks of FIG. 3, and combinations of the blocks, can be implemented by special purpose hardware-based computer that perform the specified functions, or combinations of special purpose hardware and computer instructions. In addition to the description of FIG. 3 below, the descriptions of FIGS. 1-2 are also applicable to FIG. 3, and are incorporated herein by reference. Thus, the above description may not be repeated here.

In operation 305, the first measurement device 110 measures a first biosignal. In operation 310, the first measurement device 110 transmits the first biosignal to the second measurement device 130.

In operation 315, the second measurement device 130 measures a second biosignal. In operation 320, the second measurement device 130 receives the first biosignal from the first measurement device 110. In operation 325, the second measurement device 130 transmits bioinformation including the first biosignal and the second biosignal to the drone 150.

In operation 330, the drone 150 moves to an area, for example, a area in a pen or on a farm, in which the target living body is positioned. In operation 335, the drone 150 receives the bioinformation transmitted by the second measurement device 130. The drone 150 transmits the bioinformation to the analyzer 170, in operation 340.

The analyzer 170 receives the bioinformation from the drone 150 in operation 345. In operation 350, the analyzer 170 analyzes and monitors a condition of the target living body based on the bioinformation.

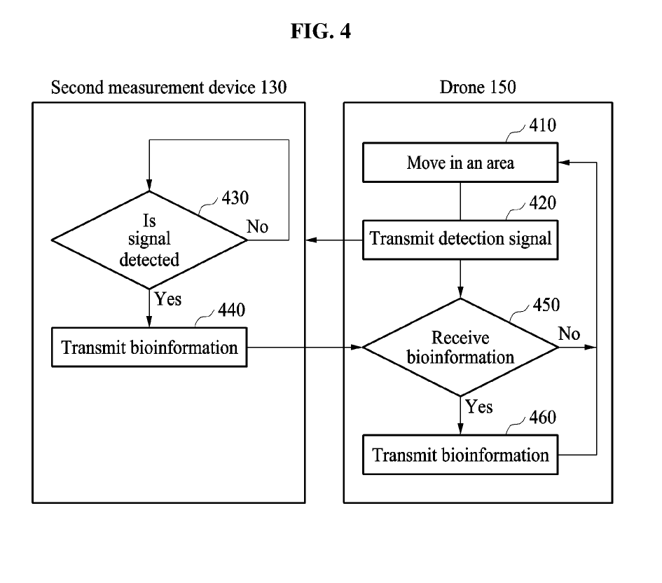

FIG. 4 is a diagram illustrating an example of a monitoring device. The operations in FIG. 4 may be performed in the sequence and manner as shown, although the order of some operations may be changed or some of the operations omitted without departing from the spirit and scope of the illustrative examples described. Many of the operations shown in FIG. 4 may be performed in parallel or concurrently. One or more blocks of FIG. 4, and combinations of the blocks, can be implemented by special purpose hardware-based computer that perform the specified functions, or combinations of special purpose hardware and computer instructions. In addition to the description of FIG. 4 below, the descriptions of FIGS. 1-3 are also applicable to FIG. 4, and are incorporated herein by reference. Thus, the above description may not be repeated here.

In operation 410, the drone 150 moves in a area in which a target living body is positioned, In operation 420, the drone 150 transmits a detection signal for verifying whether the second measurement device 130 is present.

In operation 430, the second measurement device 130 verifies whether the detection signal transmitted by the drone 150 is received. In operation 430, the second measurement device 130 verifies that the detection signal is received. In operation 440, second measurement device 130 transmits the bioinformation to the drone 150 in response to receiving the detection signal. The second measurement device 130 conserves power by transmitting the bioinformation only when receiving the detection signal from the drone 150, without unnecessarily transmitting the bioinformation.

In operation 450, the drone 150 verifies whether the bioinformation is received from the second measurement device. When the bioinformation is not received, in operation 450, the drone 150 transmits the detection signal in operation 420 by moving to the area again in operation 410.

In operation 450, when the bioinformation is received, the drone 150 transmits the bioinformation to the analyzer 170 in operation 460.

When the bioinformation is not received from the second measurement device 130, the drone 150 may iteratively transmit the detection signal in a corresponding area during a period of time.

The analyzer 170 and other apparatuses, units, modules, devices, and other components described herein are implemented by hardware components. Examples of hardware components include controllers, sensors, generators, drivers, and any other electronic components known to one of ordinary skill in the art. In one example, the hardware components are implemented by one or more processors or computers. A processor or computer is implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices known to one of ordinary skill in the art that is capable of responding to and executing instructions in a defined manner to achieve a desired result. In one example, a processor or computer includes, or is connected to, one or more memories storing instructions or software that are executed by the processor or computer. Hardware components implemented by a processor or computer execute instructions or software, such as an operating system (OS) and one or more software applications that run on the OS, to perform the operations described herein. The hardware components also access, manipulate, process, create, and store data in response to execution of the instructions or software. For simplicity, the singular term “processor” or “computer” may be used in the description of the examples described herein, but in other examples multiple processors or computers are used, or a processor or computer includes multiple processing elements, or multiple types of processing elements, or both. In one example, a hardware component includes multiple processors, and in another example, a hardware component includes a processor and a controller. A hardware component has any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 3-4 that perform the operations described in this application are performed by computing hardware, for example, by one or more processors or computers, implemented as described above executing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control a processor or computer to implement the hardware components and perform the methods as described above are written as computer programs, code segments, instructions or any combination thereof, for individually or collectively instructing or configuring the processor or computer to operate as a machine or special-purpose computer to perform the operations performed by the hardware components and the methods as described above. In an example, the instructions or software includes at least one of an applet, a dynamic link library (DLL), middleware, firmware, a device driver, an application program storing the method of preventing the collision. In one example, the instructions or software include machine code that is directly executed by the processor or computer, such as machine code produced by a compiler. In another example, the instructions or software include higher-level code that is executed by the processor or computer using an interpreter. Programmers of ordinary skill in the art can readily write the instructions or software based on the block diagrams and the flow charts illustrated in the drawings and the corresponding descriptions in the specification, which disclose algorithms for performing the operations performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media. Examples of a non-transitory computer-readable storage medium include read-only memory (ROM), random-access programmable read only memory (PROM), electrically erasable programmable read-only memory (EEPROM), random-access memory (RAM), dynamic random access memory (DRAM), static random access memory (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and any other device that is configured to store the instructions or software and any associated data, data files, and data structures in a non-transitory manner and providing the instructions or software and any associated data, data files, and data structures to a processor or computer so that the processor or computer can execute the instructions. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A monitoring device, comprising:
a first measurement device disposed inside an organ of a target, the first measurement device being configured to measure a first biosignal of the target and to transmit the first biosignal;
a second measurement device disposed inside a skin tissue of the target, and the second measurement device being configured to measure a second biosignal of the target, to receive the first biosignal, and to transmit bioinformation comprising the first biosignal and the second biosignal;
a drone configured to move to an area in which the target is positioned, to receive the bioinformation from the second measurement device, and to transmit the bioinformation; and
an analyzer configured to monitor and analyze a condition of the target based on the bioinformation received from the drone.

2. The monitoring device of claim 1, wherein the target comprises at least one of a livestock comprising a cow, a pig, a sheep, a goat, or a horse, or a pet comprising a bird, a dog or a cat.

3. The monitoring device of claim 1, wherein the organ of the target comprises at least one of a stomach, a heart, a kidney, an udder, a small intestine, a large intestine, or a rectum.

4. The monitoring device of claim 1, wherein the second measurement device is further configured to transmit the bioinformation based on an upward directivity of the target.

5. The monitoring device of claim 1, wherein the second measurement device comprises:
a second sensor configured to measure the second biosignal; and
a second communication interface configured to receive at least one of the first biosignal or a detection signal transmitted by the drone, and to transmit the bioinformation.

6. The monitoring device of claim 1, wherein the second measurement device is further configured to transmit the bioinformation to the drone, in response to receiving a detection signal transmitted by the drone.

7. The monitoring device of claim 1, wherein the first measurement device and the second measurement device are configured to communicate with each other based on any one or any combination of a human body communication method using a tissue of the target as a medium, a communication method using a 433 megahertz (MHz)-medical implant communication service (MICS) frequency band, or a near field communication (NFC) method.

8. The monitoring device of claim 1, wherein the second measurement device and the drone are configured to communicate with each other based on any one or any combination of a communication method using a 2.4 gigahertz (GHz)-industrial, scientific, and medical (ISM) frequency band including a Bluetooth and a Zigbee, a communication method using a 433 megahertz (MHz)-medical implant communication service (MICS) frequency band, or a near field communication (NFC) method.

9. The monitoring device of claim 1, wherein the drone is further configured to recognize identification information and a position of the target based on whether the bioinformation is received in the area in which the target is positioned.

10. The monitoring device of claim 9, wherein the drone is further configured to match the identification information and the position of the target to the bioinformation and to transmit the matched bioinformation to the analyzer.

11. The monitoring device of claim 1, wherein the drone is further configured to transmit a detection signal to verify a presence of the second measurement device.

12. The monitoring device of claim 11, wherein the drone is further configured to iteratively transmit the detection signal to verify a presence of the second measurement device in the area in which the target is positioned, in response to not receiving the bioinformation.

13. The monitoring device of claim 1, wherein a movement of the drone is controlled by a control signal from an outside of the monitoring device.

14. The monitoring device of claim 1, wherein the drone moves in an area based on any one or any combination of a regular pattern or a random pattern.

15. The monitoring device of claim 1, wherein the analyzer is further configured to estimate a representative biosignal of the target based on the bioinformation and to monitor a health condition of the target based on the estimated representative biosignal.

16. The monitoring device of claim 15, wherein the analyzer is further configured to:
determine the first biosignal as the representative bioinformation of the target, in response to the first biosignal being within an error range associated with the first biosignal, and
determine the second biosignal as the representative bioinformation of the target, in response to the first biosignal being outside the error range.

17. The monitoring device of claim 1, wherein the analyzer is provided integrally with the drone.

18. The monitoring device of claim 1, wherein the analyzer is provided separate from the drone.

19. The monitoring device of claim 1, wherein the first measurement device comprises:
a first sensor configured to measure the first biosignal; and
a first communication interface configured to transmit the first biosignal to the second measurement device.

20. The monitoring device of claim 1, further comprising:
a drone controller configured to control an operation including a movement of the drone.

21. An method of monitoring device a target, the method comprising:
moving a drone to an area in which the target is positioned;
transmitting a detection signal, by the drone, to verify a presence of a second measurement device in the area;
receiving bioinformation comprising a first biosignal and a second biosignal from the second measurement device, in response to the detection signal being received by the second measurement device, the first biosignal being measured by a first sensor disposed inside an organ of the target and the second biosignal being measured by a second sensor disposed inside a skin tissue of the target; and
monitoring a condition of the target based on the bioinformation.

22. A non-transitory computer-readable storage medium storing instructions that, when executed by a processor, cause the processor to perform the method of claim 21.
